(19) United States
(12) Patent Application Publication
(10) Pub. No.: US 2006/0129187 A1
(43) Pub. Date: Jun. 15, 2006

(54) Double headed tweezers

(76) Inventor: Yong Hoon Cho, Fullerton, CA (US)

Correspondence Address: Maria Erlinda C. Sarno, Esq., P.O. Box 1023, Artesia, CA, 90702, US

(21) Appl. No.: 11/012,038

(22) Filed: Dec. 14, 2004

(51) Int. Cl. A61B 17/50

(52) U.S. Cl. 606/210

(57) ABSTRACT

A double headed tweezers, comprising: a body having a pair of elongated movable pincers on one end and another pair of elongated movable pincers on an opposite end, the body formed from two flat elongated units of identical length fused or joined stationarily at a location between the opposing movable pincers. Each pincers have a tip which opens or closes according to the pressure exerted on the pincers. The tips of the pincers are shaped for a desired function. The pincers can be formed from two straight flat elongated units, from two curved elongated units or from one straight and one curved elongated units. The pincers are recommended to have a wide top surface which can be of different geometric shapes.

This invention relates to a tweezers comprising a pair of pincers or clamping mechanism on both ends, the pincers having different tips for different purpose.

### BACKGROUND

Current tweezers only have one head on a body with a stationary and a moving end. The body is generally constructed from two identical elongated thin flat sheets. The two elongated flat sheets are fused together at one end to form the stationary end resulting in a pair of free opposing ends. The fusion at one end causes the opposite end to open up or space apart, consequently, allowing movement at this end as pressure by the fingers for example, is exerted or released at the outside surfaces of the elongated flat sheets proximal to the free end.

There are different types or applications of tweezers, there are those for cosmetic purposes, usually for plucking unwanted hair, there are those used in surgery for holding or gripping tissues, blood vessels, organs and the like, there are those used in dentistry for clamping a tooth, there are those used in weaving and embroidery for plucking or inserting a thread, etc. In these applications, each tweezers has its own head. Head in this application is used to denote a pincer of the tweezers with its tips. None of these tweezers offer the flexibility of having two heads instead of one. With individual tweezers having only one head, one has to carry or prepare each individual tweezers needed. Also occasions when only one tweezers with one head is at hand, one has to adjust and make do with what one has causing sub-optimal functions.

It is therefore an object of this invention to provide a tweezers with more than one head for variable purpose or function.

It is also an object of this invention to minimize the number of individual tweezers needed to carry on a desired function or operation.

It is a further object of this invention to provide a tweezers with interchangeable heads.

### SUMMARY

This invention relates to a double headed tweezers, comprising a body having a pair of elongated movable pincers on one end and another pair of elongated movable pincers on an opposite end, the body formed from two flat elongated units of identical length fused stationarily at a location between the opposing movable pincers, each pincers having a tip, the tip opening and closing according to pressure exerted on the pincers. The tips at the end of the pincers are of various sizes and are shaped for a desired function. The double headed tweezers is fused together by welding or staking. The pincers can be formed differently, one way is from two straight flat elongated units, another, from two curved elongated units and another from one straight and one curved elongated units. A wide top and bottom surfaces of different geometric shape is recommended for the tweezers.

Instead of welding or staking a portion of the body between the opposing pincers with their corresponding tips, the double headed tweezers can have a connector having an opening at each lateral end; a first tweezers having a pincer with a tip on one end and a connecting piece on another end inserting into the opening of one lateral end of the connector; and, a second tweezers having a pincer with a tip on one end and a connecting piece on another end inserting into the opening of the other lateral end of the connector. The tips of the first and second tweezers are of various sizes and are shaped according to a desired function. The pincers are recommended to have a wide top surface of different geometric shape. The connector allows one tweezer to be replace by another tweezer thereby making the different tips of the tweezers interchangeable.

Other embodiments of the present invention will become readily apparent to those skilled in the art from the following detailed description, wherein it shows and describes only certain embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF THE DRAWINGS

Aspects of the present invention are illustrated by way of example, and not by way of limitation, in the accompanying drawings, wherein:

FIG. 1 is a perspective view of a two headed tweezers.

FIG. 2 is a perspective view of a two headed tweezers having an alternate design for the pincers.

FIG. 3 is a side view of a two headed tweezers differing in design from those shown in FIGS. 1 and 2.

FIG. 4 is a plan front view of FIG. 2.

FIG. 5A-6B are plan views of other tweezer tips.

FIG. 7 is a perspective view of the two headed tweezers of FIG. 2 joined by screws instead of fusion.

FIG. 8 is a perspective view of another way of joining two heads of the tweezers.

### DETAILED DESCRIPTION OF THE INVENTION

The detailed description represented herein is not intended to represent the only way or the only embodiment in which the claimed invention may be practiced. The description herein is provided merely as an example or examples or illustrations of the claimed invention and should not be construed as the only way or as preferred or advantageous over other embodiments or means of practicing the invention. Any tweezers with a multiple head, that is more than one in a single body is within the scope of this invention. The detailed description includes specific details to provide a thorough understanding of the claimed invention and it is apparent to those skilled in the art that the claimed invention may be practiced without these specific details. In some instances, well known structures and devices are shown in block diagrams or drawn with broken lines in order to either avoid obscuring the main concepts of the invention or to show the relationship of one part to the other.

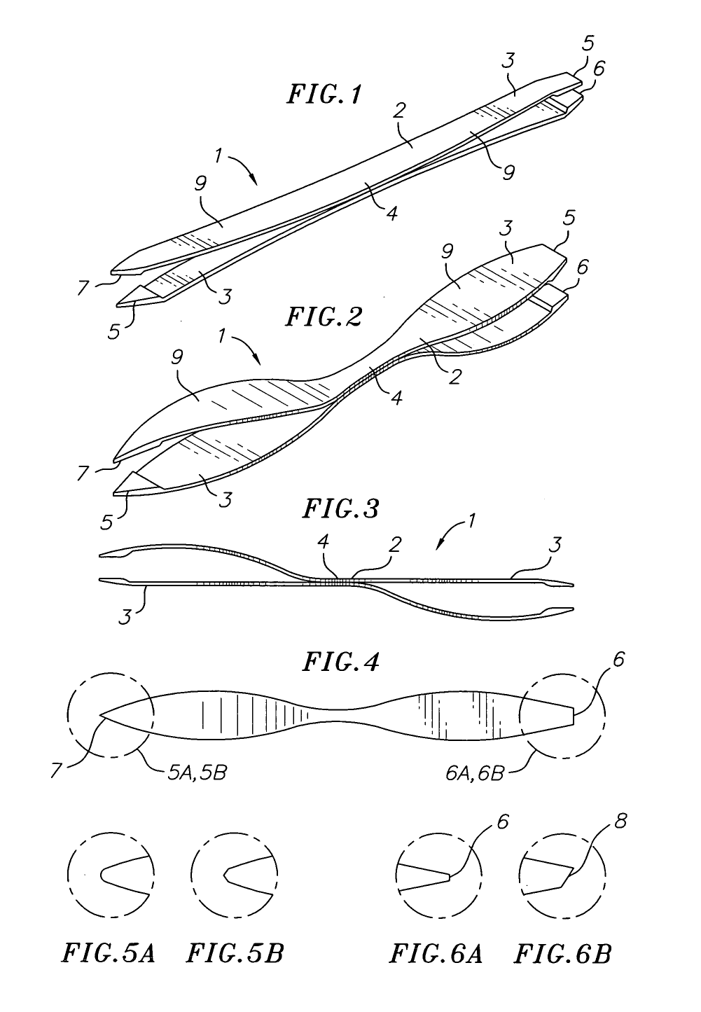

FIG. 1 is a perspective view of a two headed tweezers 1. The body 2 of the tweezers include a pair of elongated arms or pincers 3 on the opposite ends of the body 2. The pincers are fused or connected to each other at an area or a point 4 within the body 2, usually at a central location between the pincers. It is also possible to connect the pincers at another location. The pincers can have opposing tips 5 formed of any desired shape in accordance with the nature of the work to be done by the tweezers. In the claimed invention, it is recommended to have a combination of tips 5 with complimentary functions such as a square tip 6 for plucking hair from large areas of the face and a pointed tip 7 for precision shaping and plucking of short hair as shown in FIGS. 1 and 2. It is to be noted that a slanted tip 8 may be combined with either the square or pointed tip. Likewise, any tip of any shape, for example, those shown in FIGS. 5A, 5B, 6A and 6B may be combined with each other or with any of the pointed, slanted or square tip. For surgical purpose, the opposing tips may comprise tips that are straight, curved, serrated, toothed, ring shape, etc. Other fields of discipline such as dentistry may require the same or other specialized tips. The pincers 2 and the tips 5 may be designed in various sizes depending on the intended use of the tweezers.

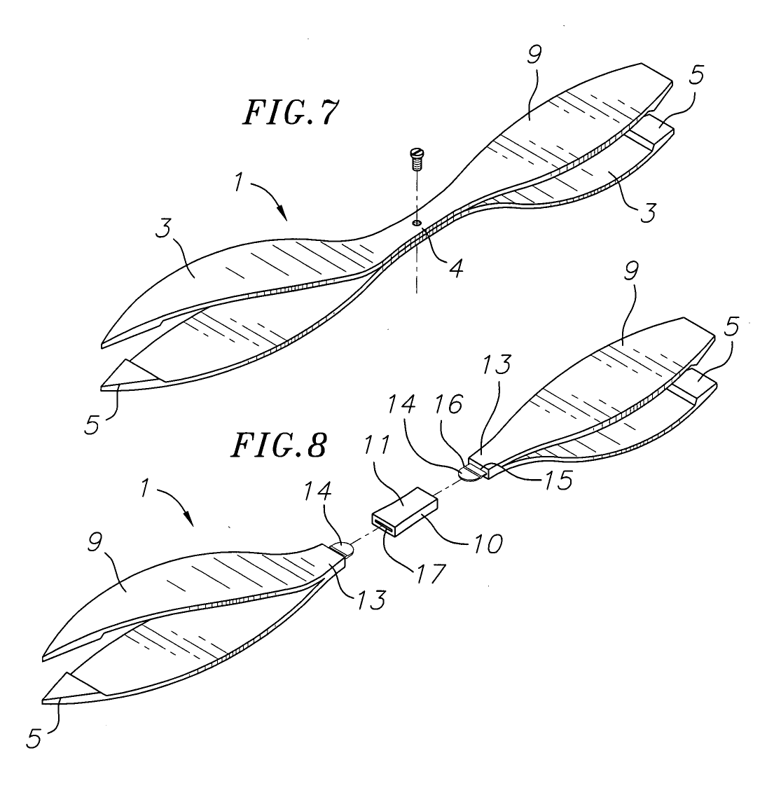

A simple way of manufacturing a two headed tweezers is to take two flat elongated units of a desired length, for example, a 4-5 inch length is sufficient to have a tweezers with two heads at both ends; cut these units to form a body 2 of the tweezers according to the desired shape of the pincers 3; shape the ends of the pincers to the desired tip 5; and fuse at a location between the opposing pincers to make the fusion point stationary while keeping the two opposing pincers free to move. The tweezers are fused together for example by welding or by staking. Other means of fusion are acceptable so long as the two elongated units are kept connected with each other, for example, the two flat elongated units forming the body of the tweezers can also be joined by screws such as that shown in FIG. 7. Materials that are currently used to make tweezers can be used for this claimed invention such as metal or metal alloys like stainless steel, titanium, chromium and others. Plastics have also been used for manufacturing tweezers. A required characteristic is to have resiliency on the pincers in order to manually operate the pincers between an open (releasing the object) and closed position (clamping the object). A user grasps the body of the tweezers anywhere along the longitudinal axis of the pincers and the pressure exerted on the tips will depend upon the distance of the tips from the source of the pressure.

As shown in FIGS. 1, 2 and 3, the pincers can have a straight or curved surface or a combination of these. FIG. 1 shows a straight pincer, FIG. 2 a curved pincer and FIG. 3, a combination of these. The curved surface offers a more aesthetic impression. A curved or concave pincer provides a feeling of greater grip to the user. The top surface 9 of the pincers 3 can be rectangular or v-shaped but it is recommended to have a different geometric shape of a wider surface such as circular, oval, oblong and the like to provide more room for the fingers thereby allowing the finger to exert more pressure without hurting the fingers. Pincers with wider top surfaces and curved peripheral edges such as that shown in FIG. 2 will also provide a greater grip during the use of the tweezers. The bottom surfaces opposite the respective top surfaces of the pincers can be shaped the same or differently than the top surfaces. A wider bottom surface is also recommended for the same reasons as stated above for the top surfaces.

With two different tips in one tweezers, one can use one tip and simply flip the tweezers and use the other tip. For example, one can pluck the hair with the square tip 6 at an area above or below the eyebrows, and then simply flip the tweezers to pluck the short hair or those on the borders to shape the eyebrows with the pointed tip 7 without putting one tweezer aside and looking for the other tweezer. Alternately, the two heads can snap in and out from two sides of a connector 10 as shown in FIG. 8. It is recommended but not required to have the top 11 and bottom surfaces (not shown) of the connector 10 planarly aligning with the top 12 and bottom surfaces (not shown) of a fused section 13 of the tweezers. As can be seen on FIG. 8, in this design, each head with the fused section looks like an individual tweezers except for the presence of a connecting piece 14 protruding from each fused end 15 of the tweezers. The connecting piece usually have a bump or bumps 16 on its surface located either on the top, bottom or the sides to provide better frictional engagement between the connector 10 and the connecting piece 14 when the connecting piece is introduced into a matching opening 17 on the connector 10. The opening is of a dimensions that would snugly fit the connecting piece. Bumps 16 may not be required if the connecting piece tightly engages with the opening on the connectors. From the illustration of how the two heads can connect to each other using connector 10, other similar types of connectors and other means of connecting the two heads can be used. The two headed tweezers result from having the connecting piece 14 on the fused end of one head inserting into one opening 17 and another connecting piece 14 on the fused end of a second head inserting into the other opening 18 of the connector 10. With a square or rectangularly shaped connector device, it is possible to have a maximum of four heads. The number of heads is dictated only by the ease and precision needed in handling the tweezers. With the use of a connector 10, it is possible to interchange the heads by simply pulling or disengaging the connecting piece from an opening of the connector and replacing this by inserting another connecting piece of another head into the same opening. By this means several heads in different combinations as desired may be used at one time.

While the embodiments of the present invention have been described, it should be understood that various changes, adaptations, and modifications may be made therein without departing from the spirit of the invention and the scope of the claims.

I claim:

1. A double headed tweezers, comprising: a body having a pair of elongated movable pincers on one end and another pair of elongated movable pincers on an opposite end, the body formed from two flat elongated units of identical length fused stationarily at a location between the opposing movable pincers, each pincers having a tip, the tip opening and closing according to pressure exerted on the pincers.

2. The double headed tweezers of claim 1 wherein the tweezers is fused at a central location between the pincers located at each end of the body.

3. The double headed tweezers of claim 1 wherein the tip is shaped for a desired function.

4. The double headed tweezers of claim 1 wherein the tips at both ends of the body have complimentary functions.

5. The double headed tweezers of claim 1 wherein the tips are of various sizes.

6. The double headed tweezers of claim 1 wherein the body is fused together by welding or staking.

7. The double headed tweezers of claim 1 wherein the body is joined together by a screw or a connector.

8. The double headed tweezers of claim 1 wherein the pincers are formed from two straight flat elongated units.

9. The double headed tweezers of claim 1 wherein the pincers are formed from two curved elongated units.

10. The double headed tweezers of claim 1 wherein the pincers are formed from one straight and one curved elongated units.

11. The double headed tweezers of claim 1 wherein the pincers have a wide top surface of different geometric shape.

12. A double headed tweezers, comprising: a connector having an opening at each lateral end; a first tweezers having a pincer with a tip on one end and a connecting piece on another end inserting into the opening of one lateral end of the connector; and, a second tweezers having a pincer with a tip on one end and a connecting piece on another end inserting into the opening of the other lateral end of the connector.

13. The double headed tweezers of claim 12 wherein the tips of the first and second tweezers are shaped for a desired function.

14. The double headed tweezers of claim 12 wherein the tips at both ends of the body have complimentary functions.

15. The double headed tweezers of claim 12 wherein the tips are of various sizes.

16. The double headed tweezers of claim 12 wherein the pincers are formed from two straight flat elongated units.

17. The double headed tweezers of claim 12 wherein the pincers are formed from two curved elongated units.

18. The double headed tweezers of claim 12 wherein the pincers are formed from one straight and one curved elongated units.

19. The double headed tweezers of claim 12 wherein the pincers have a wide top surface of different geometric shapes.

20. The double headed tweezers of claim 12 wherein the pincers with corresponding tips are interchangeable.
